(19) United States
(12) Patent Application Publication
(10) Pub. No.: US 2006/0264976 A1
(43) Pub. Date: Nov. 23, 2006

(54) System and method for improved suture insertion

(76) Inventors: Michael Allen Terry, Oak Park, IL (US); Richard James Hawkins, Spartanburg, SC (US)

Correspondence Address: MCANDREWS HELD & MALLOY, LTD, 500 WEST MADISON STREET, SUITE 3400, CHICAGO, IL, 60661, US

(21) Appl. No.: 11/135,824

(22) Filed: May 23, 2005

(51) Int. Cl. A61B 17/04

(52) U.S. Cl. 606/148

(57) ABSTRACT

Certain embodiments of the present invention provide an improved suturing system and method of use. Certain embodiments include a suturing device with a base and a hook end capable of being maneuvered to a certain position with respect to the base. The hook end may be maneuvered to a position, such as a straight position or a desired angle. The hook end may be locked at the position. The hook end may also be detachable from the base, for example. The hook end may be selected from a plurality of hook attachments having different characteristics, for example. In an embodiment, the suturing device also includes a control for maneuvering the hook end with respect to the base. In an embodiment, the suturing device further includes a locking mechanism for locking the hook end to the base.

### BACKGROUND OF THE INVENTION

The present invention generally relates to a suturing device. In particular, the present invention relates to an improved suturing device and method of use.

Many medical procedures involve suturing or joining tissue to mend tears in the tissue, protect or encourage healing or mending of bone or tissue, or repair incisions made for surgery, for example. Typically, suture needles are used to insert and tie suture material through tissue. For example, a suture needle with attached suture material may be grasped manually or using forceps and passed through tissue. The suture material may be tied in a knot using the suture needle.

Typically, suture sites are readily accessible in open surgery. However, in endoscopic surgery, which involves using an endoscope or cannula to view a body's internal structures through a small incision or puncture, a surgeon uses one or more auxiliary devices to grasp a suture needle and thread the needle through internal tissue. Surgeons are limited by reduced access space, limited vision, and limited mobility due to joints, bone, and other internal tissue. Thus, an improved device and method for suture insertion in a patient would be highly desirable.

A few attempts have been made in the prior art to improve suture needles and other suturing devices with mixed success. For example, U.S. Pat. No. 4,781,190 (Lee) discloses a method and a two-ended needle enabling arthroscopic suturing of the interior of a joint. The two-ended needle may be either straight or curved and has an eyelet midway between the ends of the needle. Alternately passing the ends of the needle through tissue to be sutured enables a suture, passing through the eyelet of the needle, to be woven through the tissue.

U.S. Pat. No. 4,441,497 (Paudler) discloses a suture passer having a plurality of flexible elongated members joined at their ends, these ends being sharpened enough to pass through desired tissue sites. Pushing the ends toward each other opens up spaces between the members into which one or more sutures may be placed. Moving the ends of the flexible members away from each other closes up the spaces to thereby grip the sutures so the ends of the flexible members and, consequently, the sutures, may be passed through desired suture sites.

Other devices known to advance suture material directly to a work site are shown in U.S. Pat. Nos. 4,890,615 (Caspari et al.) and 4,923,461 (Caspari et al.). The Caspari devices and others like them advance suture material by passing it through one or more rollers, one of which is turned by the user to propel the suture beyond the nip of the rollers. While suitable for mono-filament suture, such roller-type devices may unravel multi-stranded suture which is preferable for certain applications.

U.S. Pat. Nos. 5,279,311 (Snyder) and 5,250,053 (Snyder) discuss a suture passing device or shuttle formed of a plurality of woven or braided coated fiber strands. The Snyder devices and other prior art devices use a roller system to advance sutures through the passing device. However, when the suturing device is removed, a suture stitch may be snagged on a roller wheel and torn out of tissue. Such roller-type and wheel-based suture devices may also be difficult to utilize when simultaneously manipulating the suture device and feeding the suture material through the device into tissue.

Many prior art devices are too rigid to be manipulated internally in a patient, such as inside joints and intra-articular suturing. Additionally, Snyder and other prior art devices are too flexible to provide feeling to a surgeon during an endoscopic or other internal procedure and may potentially bend or break within the patient. Therefore, a suturing device and method with improved maneuverability and ease of use would be highly desirable.

Thus, a need exists for an improved suturing device and method of use.

### BRIEF SUMMARY OF THE INVENTION

Certain embodiments of the present invention provide an improved suturing system and method of use. Certain embodiments of an improved suturing device include a base portion and a hook portion. The base portion includes an opening at a first end for allowing suture material to be fed through the base portion. The base portion also includes an attachment location at a second end for the hook portion. The hook portion is attachable to the base portion at the attachment location. The hook portion comprises an opening for feeding suture material into a suture site, such as tissue. In an embodiment, the base portion includes an open segment for feeding the suture material in the suturing device and an enclosed segment for guiding the suture material through the base portion and the hook portion, for example.

In an embodiment, the suturing device also includes a movement mechanism for moving the hook portion with respect to the base portion. The movement mechanism includes a wheel and/or a four-bar mechanism, for example. In an embodiment, the hook portion may be maneuvered to a position, such as a straight position and/or an angle, for example. The hook portion may include a locking mechanism for locking the hook portion at the position. The hook portion may also include a locking mechanism for locking the hook portion to the base portion at the attachment location. The hook portion is selected from a plurality of hook portions, wherein each of the plurality of hook portions includes varying characteristics.

Certain embodiments of a system for improved suturing include a suturing device with a base and a hook end capable of being maneuvered to a certain position with respect to the base. The hook end may be maneuvered to a position, such as straight and/or a desired angle. The hook end may be locked at the position. The hook end may also be detachable from the base, for example. The hook end may be selected from a plurality of hook attachments having different characteristics, for example.

In an embodiment, the suturing device also includes a control for maneuvering the hook end with respect to the base. The control may include a wheel and/or a four-bar mechanism, for example. In an embodiment, the suturing device further includes a locking mechanism for locking the hook end to the base.

Certain embodiments of a method for improved suturing include selecting a hook attachment for connection to a base, feeding suture material through the base and the hook attachment, and adjusting the hook attachment to a certain position relative to the base. The feed step may also include manually feeding the suture material through the base and hook attachment. The selecting step may further include selecting the hook attachment based on one or more criteria, such as procedure, size, shape, suture site, and/or user preference.

The method may also include affixing the hook attachment to the base. In addition, the method may include stitching the suture material through tissue using the hook attachment. The method may include locking the hook attachment in a certain position. The method may also include manipulating the hook attachment through a cannula.

### BRIEF DESCRIPTION OF SEVERAL VIEWS OF THE DRAWINGS

FIG. 1 illustrates a suturing device for improved suturing in accordance with an embodiment of the present invention.

FIG. 2 shows an example of a hook locking mechanism used in accordance with an embodiment of the present invention.

FIG. 3 shows examples of a hook end used in accordance with an embodiment of the present invention.

FIG. 4 depicts examples of a hook manipulation system used in accordance with an embodiment of the present invention.

FIG. 5 illustrates a flow diagram for a method for advancing suture material through a suture site in accordance with an embodiment of the present invention.

The foregoing summary, as well as the following detailed description of certain embodiments of the present invention, will be better understood when read in conjunction with the appended drawings. For the purpose of illustrating the invention, certain embodiments are shown in the drawings. It should be understood, however, that the present invention is not limited to the arrangements and instrumentality shown in the attached drawings.

### DETAILED DESCRIPTION OF THE INVENTION

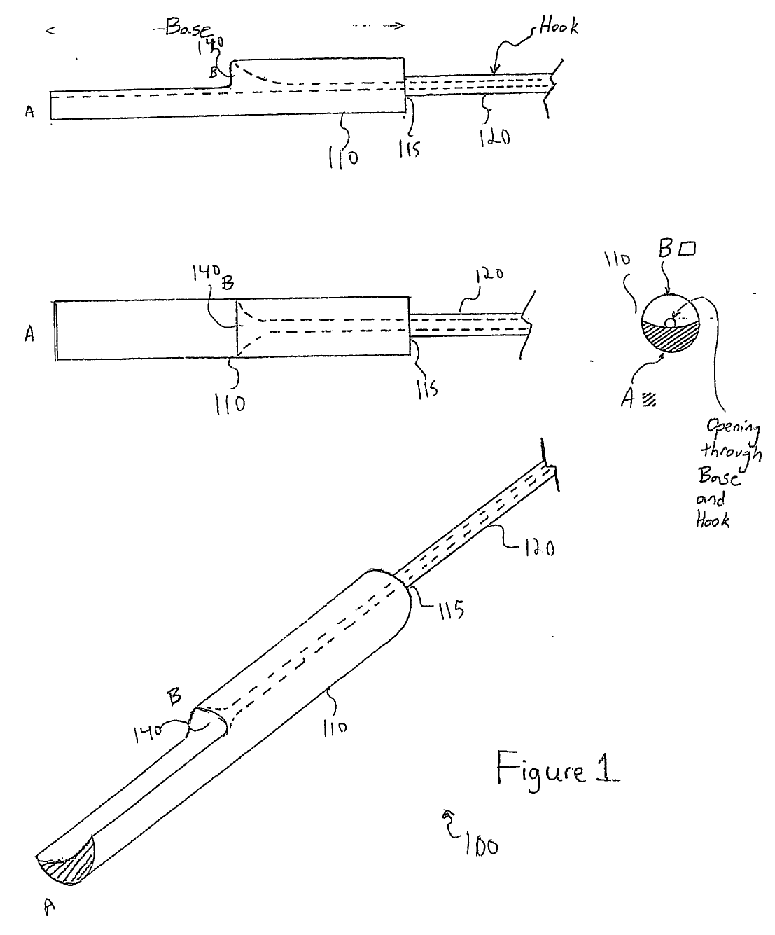

FIG. 1 illustrates a suturing device 100 for improved suturing in accordance with an embodiment of the present invention. The suturing device 100 includes a base portion 110 and a hook portion 120. The base portion 110 includes an attachment location 115 for connecting the hook portion 120 to the base portion 110. In an embodiment, the hook portion 120 and/or the base portion 110 include a locking mechanism 130 (not shown) to secure the hook portion 120 to the base portion 110 at the attachment location 115. The base portion 110 and hook portion 120 may include openings 140, 145 (not shown) to allow material, such as suture material, to pass through the suturing device 100. The suturing device 100 may be constructed from metal, plastic, and/or composite material, for example. In an embodiment, the suturing device 100 may be made from a material such as a metal to resist bending while in a joint. In an embodiment, the base portion 110 may be made with a plastic portion on which a user's thumb or other digit may slide to push suturing material into the hook portion 120. In an alternative embodiment, the base portion 110 and hook portion 120 may be integrated into a single unit as well as separated.

In an embodiment, one of a plurality of hook portions 120 may be selected for attachment to the base portion 110. The base portion 110 connects temporarily to one of a plurality of hook portions 120. That is, various hook portions 120 may be replaced at the attachment location 115 on the base portion 110 based on certain criteria, such as user preference, procedure, suture site, size and/or shape limitation, etc.

The hook portion 120 connects to the base portion 110 at the attachment location 115. For example, the hook portion 120 may snap or slide into or onto the base portion 110 at the attachment location 115. Alternatively, the hook portion 120 may screw onto the base portion 110 at the attachment location 115. The attachment location 115 is located at an end of the base portion 110, such as a proximal end of the base portion 110, for example. Additional methods to secure the hook portion 120 to the base portion 110 may be used.

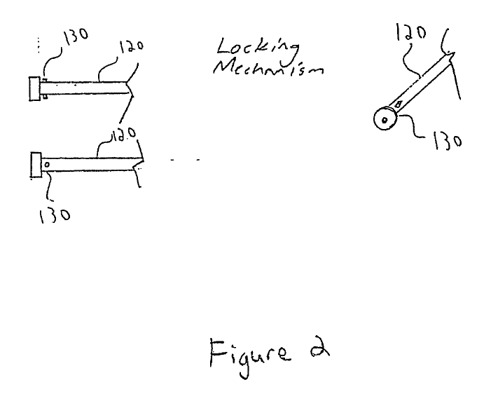

In an embodiment, the locking mechanism 130 may be used to lock the hook portion 120 into the base portion 110 at the attachment location 115. For example, as shown in FIG. 2, the locking mechanism 130 may include one or more protrusions that lock into grooves or openings at the attachment location 115 upon rotating (e.g., rotating the hook portion 120 ninety degrees relative to the base portion 110) or inserting the hook portion 120 into the base portion 110 at the attachment location 115. Alternatively or in addition, the locking mechanism 130 may include a clasp, snap, locking ring, and/or other locking device, for example.

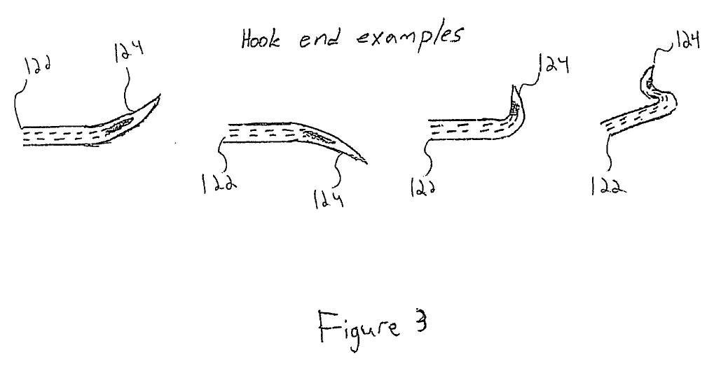

In an embodiment, the hook portion 120 includes a base end 122 and a hook end 124. The hook portion 120 may also include an opening 145 that extends through the hook portion 120 to allow suture material or other material to pass through the hook portion 120. The base end 122 allows the hook portion 120 to be attached to the base portion 110 at the attachment location 115. The hook end 124 includes a tip that may be bent or curved at a certain angle and/or arc, for example. The hook end 124 may also be a straight end, for example. Some hook end 124 examples are shown in FIG. 3.

Thus, a plurality of hook portions 120 having one or more different characteristics may be available for attachment to the base portion 110 via the attachment location 115. For example, hooks with a variety of different angles may be available for attachment to the base portion 110. In addition, different radii of curvature may be described by providing a plurality of hooks with varying radius of curvature, arc of curvature, angle, or any of the above in combination, for example. In an embodiment, a user or other system may select an appropriate hook portion for an operative site. Sites of operation may differ in a plurality of ways including: laterality, location within the operative cavity or joint, tissue to be manipulated, etc.

Suture material or other material may be fed through the suturing device 100 via the openings 140, 145 in the base portion 110 and hook portion 120. The openings 140, 145 provide a passage through the suturing device 100 through which the suture material may travel. Suture material may slide into the hook portion 120 from the base portion 110. Suture material may be pushed or fed manually, for example, over a flat section of the base portion 110 prior to the opening 140. The suture material is then pushed into a passage formed by the opening 140 leading to the opening 140 of the hook portion 120. The hook portion 120 may then be used to insert or stitch the suture material through tissue or other material, for example.

In an embodiment, the base portion 110 includes a first segment, such as a proximal segment, that is open or uncovered (e.g., a segment of the base portion 110 from point A to point B as shown in FIG. 1). That is, the first segment of the base portion 110 is open to allow suture material or other material to be advanced, such as manually advanced by a user, into the opening 140 and through the base portion 110 and hook portion 120 into a suture site, such as a patient wound. A second segment of the base portion, such as a distal segment, is enclosed to form a cylinder with opening 140 to guide the suture material or other material through the base portion 110 and the hook portion 120, for example.

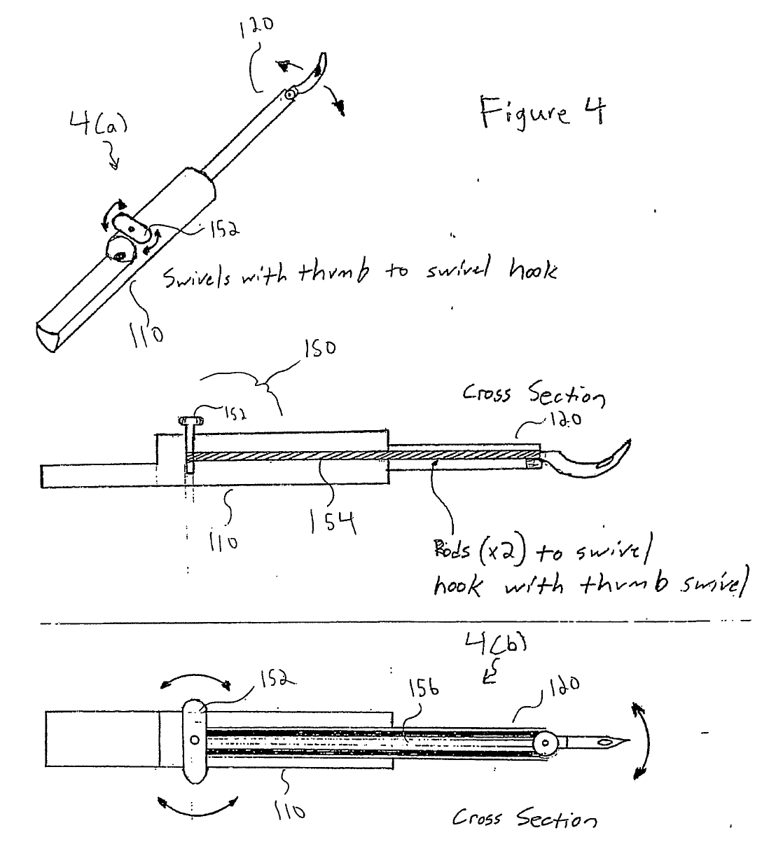

In an embodiment, the base portion 110 includes a manipulator 150 or other control for manipulating the hook portion 120 relative to the base portion 110. Examples of a manipulator system 150 are depicted in FIG. 4. For example, as shown in FIG. 4(a), a swivel 152 and rod 154 system may be used to move the hook end 124 of the hook portion 120 in a plurality of directions. An amount of rotation applied to the swivel 152 determines a degree of movement of the hook end 124, for example. The system may include one or more rods 154 to allow movement of the hook end 124 in a plurality of directions using the swivel 152 (e.g., one or more wheel(s), bar(s), button(s) and/or other swivel(s)). As shown in FIG. 4(b), a four-bar mechanism 156 may be used to rotate or otherwise maneuver the hook end 124 or entire hook portion 120, for example. In an embodiment, the manipulator mechanism 150 may be locked to prevent unwanted movement of the hook end 124 or hook portion 120 with respect to the base portion 110. The manipulator mechanism 150 may lock the hook end 124 and/or hook portion 120 at any selected angle and/or arc, as well as in a straight position, for example.

In operation, the suturing device 100 may be maneuvered into a joint. The suturing device 100 may be kept rigid to maneuver the device 100 into the joint. Then, a suture may be passed through tissue using the suturing device 100. The suturing device 100 may be used to pierce the tissue and push a suture through the tissue. The suture is then grasped at an exit end of the tissue and manipulated to complete the suturing process as appropriate.

In an embodiment, sutures are advanced or “fed” through the suturing device 100 for stitching in tissue by a user's digit, such as a finger (e.g., a thumb). Use of a digit, such as a thumb, helps sutures to be advanced more quickly and helps the suturing device 100 to be withdrawn more smoothly and maneuvered more stoutly than other suturing devices. For example, a surgeon's thumb holds a suture in the device 100. Then, the surgeon pulls the device 100 out of the suture site. The surgeon may pull the suture stitch and tie the stitch or connect the stitch to another stitch, for example. The device 100 may be removed with little friction on the stitch rather than using one or more wheels to feed the suture material that may snag when the suturing device 100 is removed.

Unlike prior art devices, where right angles or “hard” angles are difficult to maneuver, certain embodiments enable the suturing device 100 to be maneuvered around tight corners, such as right angles or other hard angles (e.g., sixty degree angles). Additionally, prior art devices often utilize a hook with a short penetration portion, which is not very useful for suturing. For example, if a prior art suturing device is used to turn anything other than a few degrees, the short hook is not useful and is difficult to maneuver through a cannula. Certain embodiments of the present invention provide a variety of hook attachments of varying sizes, shapes, and configurations to satisfy a variety of functions and situations, for example.

Conversely, certain embodiments provide a suturing device 100 that may be inserted straight and turned once the device 100 is inside a desired area, such as a joint. The device 100 may be straightened to travel through a cannula and modified or turned once the device 100 is inside. To remove the device 100, the device 100 may be straightened for ease of removal. In an embodiment, the device 100 may include a setting mechanism, such as a wheel or four-bar mechanism, to set the angle of the hook portion 120 with respect to the base portion 110.

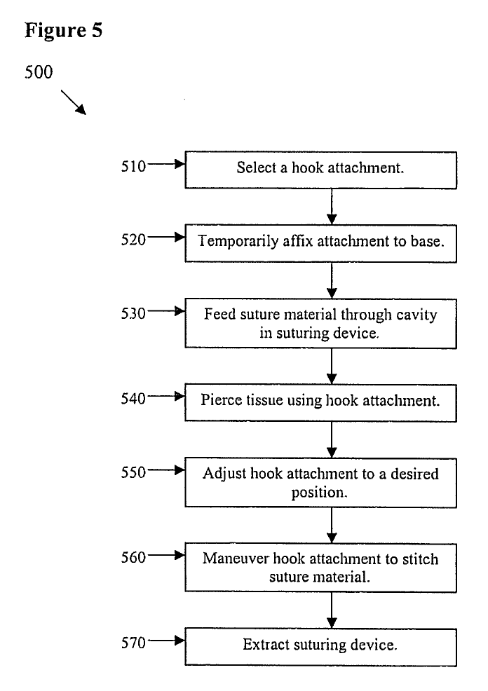

FIG. 5 illustrates a flow diagram for a method 500 for advancing suture material through a suture site in accordance with an embodiment of the present invention. First, at step 510, a hook attachment is selected. For example, a hook attachment may be selected for an endoscopic surgical procedure based on a desired hook shape. Next, at step 520, the hook attachment is temporarily affixed to a base. For example, a receptacle in the base allows one of a plurality of hook attachments to be temporarily connected to the base.

Then, at step 530, suture material is fed through a cavity in the base and hook attachment. For example, a surgeon feeds suturing material through the cavity in the suturing device using a thumb or other convenient digit. A user may continue to feed suture material through the cavity and into a patient at a suture site as the base and hook attachment are manipulated to stitch at the suture site, such as to close a wound or tear in tissue at the suture site.

At step 540, the hook attachment is used to pierce a patient's tissue to allow suture material to penetrate the tissue. For example, a surgeon manipulates the rigid hook through tissue to a target suture site. The hook attachment may be positioned in a rigid or straight position with respect to the base to allow the surgeon to negotiate within a joint and tissue to a target suture site, for example. Then, at step 550, the hook attachment is adjusted to a desired position in relation to the base. For example, a four-bar and swivel mechanism may be used by a surgeon to position the hook attachment at a desired angle and direction with respect to the base. The hook attachment may be manipulated to stitch suture material at a suture site.

At step 560, the hook attachment may be maneuvered as desired to stitch suture material at a suture site on or in a patient. For example, a surgeon may angle the hook attachment or hook end of the hook attachment relative to the base to maneuver the hook through the tissue via a desired path at a suture site. The hook may be manipulated to maneuver through tissue and around bone in a patient, for example. The suture is then shuttled through the base and hook into the cavity or joint.

Then, at step 570, the suturing device is extracted from the patient. For example, the hook attachment may be locked in a strait position or angled to move through and/or around bone and/or tissue to extract the suturing device from the patient. The suture, which was left traversing the desired tissue, is then grasped using a second device, for example, for final manipulation to complete the suturing process. An alternative involves passing a device called a suture shuttle or relay, for example, into the cavity or joint using the base 100 and hook 120 and pulling a suture through the tissue traversed by the hook 120 secondarily from outside the joint or cavity to inside the joint or cavity. Another possibility involves passing a suture shuttle or relay, for example, into the joint or cavity and connecting that to a suture that is in the joint or cavity to pull the suture out through the tissue traversed by the hook 120.

The steps of the method 500 may be performed in a variety of combinations or orders, for example. Additionally, one or more of the steps of the method 500 may be eliminated. Furthermore, additional steps may be added to the execution of the method 500.

Thus, certain embodiments provide a suturing device for use in a variety of medical procedures. Certain embodiments may be used for tissue suturing, intra-articular sutures, and/or joint-related suturing operations, for example. For example, certain embodiments may be used for tissue tears, shoulder sutures, rotator cuff surgery, miniscal tear repair, and other procedures. Certain embodiments provide a suturing device capable of being manipulated within a joint for intra-joint procedures as well as tissue-based suturing. For example, certain embodiments provide a suturing device with a hook attachment capable of being locked in a straight position and/or in a plurality of angled/arced positions for manipulation in and/or around bone and/or tissue.

Certain embodiments provide a suture device with a plurality of replaceable hook attachments. A user may select one of the plurality of hook attachments to connect to a base for suturing. A hook attachment may be selected based on criteria such as size/shape limitations, procedure, suture site, and/or other characteristic, for example. Certain embodiments provide a hook attachment that may be moved, positioned, or otherwise manipulated to provide a user with increase flexibility and mobility in moving the suturing device for suturing.

While the invention has been described with reference to certain embodiments, it will be understood by those skilled in the art that various changes may be made and equivalents may be substituted without departing from the scope of the invention. In addition, many modifications may be made to adapt a particular situation or material to the teachings of the invention without departing from its scope. Therefore, it is intended that the invention not be limited to the particular embodiment disclosed, but that the invention will include all embodiments falling within the scope of the appended claims.

1. An improved suturing device, said suturing device comprising:
a base portion, wherein said base portion comprises an opening at a first end for allowing suture material to be fed through said base portion; and
a hook portion attachable to said base portion, wherein said hook portion comprises an opening for feeding said suture material into a suture site.

2. The suturing device of claim 1, further comprising a movement mechanism for moving said hook portion with respect to said base portion.

3. The suturing device of claim 2, wherein said hook portion may be maneuvered to a position.

4. The suturing device of claim 3, further comprising a locking mechanism for locking said hook portion at said position.

5. The suturing device of claim 1, wherein said hook portion is selected from a plurality of hook portions, wherein each of said plurality of hook portions includes varying characteristics.

6. The suturing device of claim 1, wherein said hook portion further comprises a locking mechanism for locking said hook portion to said base portion.

7. The suturing device of claim 1, wherein said base portion further comprises an open segment for feeding said suture material and an enclosed segment for guiding said suture material through said base portion and said hook portion.

8. A system for improved suturing, said system comprising:
a suturing device comprising a base and a hook end capable of being maneuvered to a certain position with respect to said base.

9. The system of claim 8, wherein said hook end may be maneuvered to a position.

10. The system of claim 9, wherein said hook end may be locked at said position.

11. The system of claim 8, wherein said suturing device further comprises a control for maneuvering said hook end with respect to said base.

12. The system of claim 8, wherein said hook end is detachable from said base.

13. The system of claim 12, wherein said suturing device further comprises a locking mechanism for locking said hook end to said base.

14. The system of claim 12, wherein said hook end is selected from a plurality of hook attachments having different characteristics.

15. A method for improved suturing, said method comprising:
selecting a hook attachment for connection to a base;
feeding suture material through said base and said hook attachment; and
adjusting said hook attachment to a certain position relative to said base.

16. The method of claim 15, wherein said feeding step further comprises manually feeding said suture material.

17. The method of claim 15, wherein said selecting step further comprises selecting said hook attachment based on a criterion.

18. The method of claim 17, wherein said criterion comprises at least one of a procedure to be performed, a size limitation, a shape limitation, a suture site, and a user preference.

19. The method of claim 15, further comprising affixing said hook attachment to said base.

20. The method of claim 15, further comprising stitching said suture material through tissue using said hook attachment.

21. The method of claim 15, further comprising locking said hook attachment in a certain position.
